(19) United States
(12) Patent Application Publication
(10) Pub. No.: US 2005/0158802 A1
(43) Pub. Date: Jul. 21, 2005

(54) UbcH8 Ubiquitin E2 enzyme is also the E2 enzyme for ISG15, an interferon alpha/beta induced Ubiquitin-like protein

(75) Inventors: Robert M. Krug, Austin, TX (US); Chen Zhao, Austin, TX (US); Jon Huibregtse, Austin, TX (US)

Correspondence Address: CHALKER FLORES, LLP, 12700 PARK CENTRAL, STE. 455, DALLAS, TX, 75251, US

(73) Assignee: Board Of Regents, The University of Texas System, Austin, TX (US)

(21) Appl. No.: 11/020,853

(22) Filed: Dec. 22, 2004

Related U.S. Application Data
(60) Provisional application No. 60/532,043, filed on Dec. 23, 2003.

(51) Int. Cl. G01N 33/53; G01N 33/567; C12Q 1/37; C12N 9/64; A61K 38/17

(52) U.S. Cl. 435/7.2; 435/226; 435/23; 514/2

(57) ABSTRACT

The present invention relates to compositions and methods for the modulation of both ubiquitin and ubiquitin-like protein mediated modification and degradation of target proteins that includes the steps of contacting the compound with an Ubiquitin-like protein E2 enzyme and an E1 enzyme containing an ISG15 protein and an E1 containing ubiquitin and detecting the effect of the compound on the interaction between the Ubiquitin-like protein E2 enzyme and these two E1 enzymes, and the steps of contacting the compound with an Ubiquitin-like E2 enzyme containing an ISG15 protein or the same E2 enzyme containing ubiquitin and the E3 enymes that function with this E2 enzyme and detecting the effect of the compound on the interaction between these E2 and E3 enzymes.

This application claims priority to Provisional Application Ser. No. 60/532,043 filed Dec. 23, 2003.

The U.S. Government may own certain rights in this invention pursuant to the terms of the NIH Grant No. All 7772 and CA72943.

### TECHNICAL FIELD OF THE INVENTION

The present invention relates in general to the field of protein modification and degradation and, more particularly, to compounds that modulate an enzyme complex that is able to conjugate both ubiquitin and ubiquitin-like proteins to protein targets, thereby regulating the functions and degradation of the protein targets.

### BACKGROUND OF THE INVENTION

Without limiting the scope of the invention, its background is described in connection with the modification and degradation of proteins.

Heretofore in this field, it has been recognized that the proper development and maintenance of a multicellular organism is a complex process that requires precise spatial and temporal control of cell proliferation. For example, cell proliferation is controlled via an intricate network of extracellular and intracellular signaling pathways that process growth regulatory signals. These signaling pathways are superimposed upon the basic cell cycle regulatory machinery that controls particular cell cycle transitions. In eukaryotes, the cell cycle includes an ordered series of discrete events.

In contrast to the periodicity of eukaryotic DNA replication and mitosis, cellular growth requires that most metabolic reactions occur continuously. Both the continuous metabolic maintenance of the cell and cell cycle regulation require precise control of protein degradation. The control of cell division requires one or more signals that initiate the cell cycle, induce DNA replication or mitosis and eventually lead to cell division upon reaching a critical concentration. The mitotic process inactivates the initiator, thereby “resetting” the cell cycle. Mitotic cyclins accumulate during interphase to drive entry of cells into mitosis and are then degraded at the end of mitosis, thereby resetting the cell cycle. Protein degradation via post-translational addition of Ubiquitin to target proteins plays a key role in the regulation of cell cycle progression. For example, proteolysis is required for multiple mitotic processes, and for initiating DNA replication.

Nonetheless, much remains unknown regarding the proteins and the interactions that are involved in the non-proteolytic regulation of Ubiquitin-like proteins of the cell cycle and other processes. Indeed, many proteins are likely to be involved in proteolysis and cellular maintenance.

### SUMMARY OF THE INVENTION

The present invention involves the post-translational, covalent conjugation of ubiquitin (Ub) or ubiquitin-like proteins (Ubls) is a major eukaryotic regulatory mechanism. Conjugation of Ub requires an enzymatic cascade involving an E1 activating enzyme, E2 conjugating enzymes, and E3 ligases. The conjugation pathways of several Ubls have been shown to parallel the Ub pathway, but to use different E1, E2 and E3 enzymes. Both the synthesis of the Ubl ISG15 and its conjugation to proteins are induced by interferon. The only characterized component of the ISG15 conjugation cascade is the E1 enzyme, Ube1 L, which activates ISG15, but not Ub.

More particularly, the present invention includes compositions and methods that involve the isolation, identification, and characterization of the Ubl E2 enzyme that functions in the ISG15 conjugation pathway. One example of the Ubl E2 enzyme found is UbcH8, a human E2 enzyme that has been shown to function in Ub conjugation. Further, the inventors demonstrated that two HECT E3 enzymes that function with UbcH8 in Ub conjugation can also function with UbcH8 in ISG15 conjugation. Thus, the pathways for Ub and ISG15 conjugation converge downstream of the E1 enzymes, at the UbcH8 E2 enzyme. This convergence of proteins that are involved in post-translational, covalent conjugation of Ub and Ubls allows for the identification of compounds that may modulate the effects of activation of both the Ub and Ubl pathways. Furthermore, the invention relates to compounds that may affect the conjugation of ISG15 as regulated by interferon.

More particularly, the present invention include a method of selecting a compound that modulates the conjugation of a Ubiquitin and/or a Ubiquitin-like proteins to a target protein comprising the step of contacting the compound with a Ubiquitin-like protein conjugating enzyme that conjugates a Ubiquitin or a Ubiquitin-like protein to the target protein and detecting the effect of the compound on the Ubiquitin-like protein conjugating enzyme. One example of a Ubiquitin-like protein conjugating enzyme that is able to conjugate with both Ub and Ubls is UbcH8. Another example of the Ubiquitin-like protein conjugating enzyme is an interferon-inducible ubiquitin conjugation E2 complex, e.g., wherein the Ubiquitin-like protein conjugating enzyme is upregulated by interferon. The method may further include the step of adding a complete protein degradation proteosome and detecting the level of the target protein in the presence or absence of the compound.

A wide variety of methods of detecting the interaction between an E1, E2 and an E3 protein and Ub or Ubls for use with the present invention includes detecting, e.g., protein degradation, protein binding, protein labeling, protein presence, ubiquitin conjugation, ubiquitin-like protein conjugation, thioester-linkage between the Ubiquitin and Ubiquitin-like protein and the Ubiquitin-like protein conjugating enzyme and combinations thereof. In one embodiment the step of contacting is performed in vitro, in vivo using cell extracts, using purified components and the like. The Ubiquitin-like protein conjugating enzyme may even be an isolated and purified fusion UbcH8 protein, e.g., a GST-UbcH8. The Ubiquitin or a Ubiquitin-like protein may also be an isolated and purified Ubiquitin-like fusion protein, e.g., a Ubiquitin or a Ubiquitin-like protein comprises GST-ISG15.

Yet another method of the present invention includes selecting a compound that modulates the conjugation of Ubiquitin-like proteins to target proteins that includes the steps of contacting the compound with an Ubiquitin-like protein E2 enzyme and the Ubiquitin-like E1 enzyme comprising the ISG15 protein and detecting the effect of the compound on the interaction between the Ubiquitin-like protein E2 enzyme and the Ubiquitin-like E1 enzyme comprising the ISG15 protein. The Ubiquitin-like protein E2 enzyme comprises UbcH8, e.g., an interferon-inducible ubiquitin conjugation E2 complex. The interferon will generally be a type I interferon, e.g., interferon alpha, interferon beta or mixtures thereon.

Yet another method of the present invention includes selecting a compound that modulates both the degradation of Ubiquitin and Ubiquitin-like proteins by contacting the compound with the E2 enzyme HbcH8 and an ISG15 protein and detecting the effect of the compound on the interaction between the HbcH8 and the ISG15 proteins. The Ubiquitin-like protein E2 enzyme may use a GST-UbcH8 and an ISG15, e.g., GST-ISG15. The effect on the target protein detected may be by detecting the degradation via the Ubiquitin-like protein pathway and detecting the level of the target protein in the presence or absence of the compound.

Yet another embodiment of the present invention is a high throughput assay for selecting a compound that modulates the conjugation of Ubiquitin and Ubiquitin-like proteins to a target protein by contacting one or more compounds with a Ubiquitin-like protein conjugating enzyme that conjugates a Ubiquitin or a Ubiquitin-like protein to a target protein, detecting the effect of the compound on the Ubiquitin-like protein conjugating enzyme and isolating the one or more compounds that affect the activity of the Ubiquitin-like protein conjugating enzyme.

The present invention also includes modulators or inhibitors of the ubiquitin-like protein degradation identified by the method disclosed herein. For example, the inhibitor of ubiquitin-like protein and ubiquitin degradation includes one or more molecules that interfere with the binding of ISG15 and UbcH8 and/or one or more molecules that interfere with the binding of Ubiquitin and UbcH8.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are coomasie blue stained polyacrylamide gels that show the isolation of the E2 protein that binds ISG15;

FIGS. 2A, 2B and 2C are gels in which: (2A) ³²P labeled E2-GST-ISG15 binding to UbcH8 is demonstrated; (2B) the specificity of binding to UbcH8 is demonstrated; and (2C) northern gel (left panel) and protein binding after siRNA interference (right panel) to UbcH8;

FIGS. 3A, 3B, 3C and 3D are gels that demonstrate function and interaction between UbcH8 and HECT E3s, namely: (3A) ISG15 thioester formation; (3B) HPV E6/E6AP-dependent conjugation of ISG15 to p53; (3C) ³²P-ISG15 binding to p53; and (3D) the specificity of purified FLAG-WBP2 binding to ISG15.

FIG. 4 is a diagram that summarizes the role of the E2 Ubiquitin-like protein conjugating enzyme.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Ubiquitin-like protein conjugating enzyme” and “E2 Ubiquitin-like protein conjugating enzyme,” is conjugating enzyme that conjugates a Ubiquitin or a Ubiquitin-like protein to a target protein. As the skilled artisan will know, one or more Ubiquitins are attached to target proteins post-translationally through a complex of three enzyme or enzyme complexes: Ubiquitin-specific E1, E2 and E3, which lead to protein degradation. A non-overlapping complex has been theorized for Ubls. The present inventors have not only isolated, purified and characterized the first E2 protein but disclose herein the first cross-over between the Ub and Ubl pathways. Furthermore, the Ubiquitin-like protein conjugating enzyme provides a target for the isolation, characterization, development and testing of molecules that enhance, modify, reduce or eliminate the activity of the Ubiquitin-like protein conjugating enzyme of the present invention.

As used herein, the term “multiprotein complex” refers to complexes comprising more than one protein. It is intended that the term encompass complexes with any number of proteins. In one embodiment, the proteins comprising a multiprotein complex function cooperatively. It is also intended that the term encompass complexes of cross-reacting proteins of the E1, E2 and E3 protein complexes.

The terms “modulate” and “modify” as used herein, refers to a change or an alteration in the biological activity of the “Ubiquitin-like protein conjugating enzyme” and “E2 Ubiquitin-like protein conjugating enzymes,” including changes in the level of mRNA, translation and even post-translational modifications. Modulation may be an increase or a decrease in protein activity, a change in binding characteristics, or any other change in the biological, functional, or immunological properties of the “Ubiquitin-like protein conjugating enzyme” and “E2 Ubiquitin-like protein conjugating enzymes.”

The term “mimetic,” as used herein, refers to a molecule, the structure of which is developed from knowledge of the structure of the “Ubiquitin-like protein conjugating enzyme” and “E2 Ubiquitin-like protein conjugating enzymes,” or portions thereof and, as such, is able to effect some or all of the actions of Ubl-like molecules.

The term “antagonist” refers to molecules or compounds which inhibit the action of a composition (e.g., the “Ubiquitin-like protein conjugating enzyme” and “E2 Ubiquitin-like protein conjugating enzymes,” and UbcH8). Antagonists may or may not be homologous to the targets of these compositions in respect to conformation, charge or other characteristics. In one embodiment, antagonists prevent the functioning of “Ubiquitin-like protein conjugating enzyme” and “E2 Ubiquitin-like protein conjugating enzymes.” Antagonists may prevent binding of an “Ubiquitin-like protein conjugating enzyme” and “E2 Ubiquitin-like protein conjugating enzymes,” and its target(s). It is also contemplated that antagonists prevent or alter the binding of an UbcH8 and ISG15. However, it is not intended that the term be limited to a particular site of function.

The term “derivative,” as used herein, refers to the chemical modification of a nucleic acid encoding an “Ubiquitin-like protein conjugating enzyme” and/or a “E2 Ubiquitin-like protein conjugating enzymes,” (in particular, UbcH8), or the encoded protein. Illustrative of such modifications would be replacement of hydrogen by an alkyl, acyl, or amino group. A nucleic acid derivative would encode a polypeptide which retains essential biological characteristics of the natural molecule.

A “variant” of an “Ubiquitin-like protein conjugating enzyme” and “E2 Ubiquitin-like protein conjugating enzymes,” as used herein, refers to an amino acid sequence that is altered by one or more amino acids. The variant may have “conservative” changes, wherein a substituted amino acid has similar structural or chemical properties (e.g., replacement of leucine with isoleucine). More rarely, a variant may have “nonconservative” changes (e.g., replacement of a glycine with a tryptophan). Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

“Alterations” in the polynucleotide of the “Ubiquitin-like protein conjugating enzyme” and “E2 Ubiquitin-like protein conjugating enzymes,” include any alteration in the sequence of polynucleotides encoding “Ubiquitin-like protein conjugating enzyme” and “E2 Ubiquitin-like protein conjugating enzymes,” including deletions, insertions, and point mutations that may be detected using hybridization assays. Included within this definition is the detection of alterations to the genomic DNA sequence that encodes a “Ubiquitin-like protein conjugating enzyme,” “E2 Ubiquitin-like protein conjugating enzymes,” or even HbcH8 (e.g., by alterations in the pattern of restriction fragment length polymorphisms) capable of hybridizing to a particular sequence, the inability of a selected fragment to hybridize to a sample of genomic DNA (e.g., using allele-specific oligonucleotide probes), and improper or unexpected hybridization, such as hybridization to a locus other than the normal chromosomal locus for the polynucleotide sequence encoding an UbcH8 (e.g., using fluorescent in situ hybridization [FISH] to metaphase chromosomes spreads).

The term “sample,” as used herein, is used in its broadest sense. The term encompasses biological sample(s) suspected of containing “Ubiquitin-like protein conjugating enzyme” and “E2 Ubiquitin-like protein conjugating enzymes,” or fragments thereof, and include, e.g., a cell, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), an extract from cells or a tissue, and the like.

As used herein the terms “protein” and “polypeptide” refer to compounds comprising ammo acids joined via peptide bonds and are used interchangeably. The term “portion,” as used herein, with regard to a protein (as in “a portion of a given protein”) refers to fragments of that protein. The fragments may range in size from four amino acid residues to the entire amino acid sequence minus one amino acid. Thus, a protein “comprising at least a portion of the amino acid sequence of an “Ubiquitin-like protein conjugating enzyme” and “E2 Ubiquitin-like protein conjugating enzymes,” or “UbcH8” encompasses the full-length human protein, and fragments thereof.

The term “biologically active,” as used herein, refers to a protein having structural, regulatory, or biochemical functions of a naturally occurring molecule. Likewise, “immunologically active” refers to the capability of the natural, recombinant, or synthetic “Ubiquitin-like protein conjugating enzyme” and “E2 Ubiquitin-like protein conjugating enzymes,” or any oligopeptide thereof, to induce a specific immune response in appropriate animals or cells and to bind with specific antibodies.

As used herein, the term “purified” or “to purify” refers to the removal of contaminants from a sample. For example, proteins of interest are purified by isolation as fusion proteins, removal of contaminating proteins and/or purified by the removal of substantially all proteins that are not of interest. The removal of non-immunoglobulin proteins and/or the removal of immunoglobulins that do not bind protein results in an increase in the percent of protein of interest-reactive immunoglobulins in the sample. In another example, recombinant polypeptides are expressed in bacterial host cells and the polypeptides are purified by the removal of host cell proteins, thereby increasing the percent of recombinant polypeptides in the sample. The term “substantially purified,” as used herein, refers to nucleic or amino acid sequences that are removed from their natural environment, isolated or separated, and are at least 60% free, 75% free, and even 90% free from other components with which they are naturally associated.

A “host cell” is any cell that is able to carry, transiently or permanently a nucleic acid segment that encodes a “Ubiquitin-like protein conjugating enzyme” and “E2 Ubiquitin-like protein conjugating enzymes,” and mey even express the protein.

As used herein, the term “fusion protein” refers to a chimeric protein containing the protein of interest (i.e., “Ubiquitin-like protein conjugating enzyme” and “E2 Ubiquitin-like protein conjugating enzymes,” “UbcH8”) joined to one or more exogenous protein fragment as will be know to those of skill in the art and as taught herein. The fusion partner may enhance solubility of the protein as expressed in a host cell, may provide an “affinity tag” to allow purification of the recombinant fusion protein from the host cell or culture supernatant, or both. If desired, the fusion protein may be removed from the protein of interest prior to immunization by a variety of enzymatic or chemical means known to the art. As used herein, the term “affinity tag” refers to such structures as a “poly-histidine tract” or “poly-histidine tag,” “FLAG,” “GST” or any other structure or compound which facilitates the purification of a recombinant fusion protein from a host cell, host cell culture supernatant, or both. As used herein, the term “FLAG tag” refers to short polypeptide marker sequence useful for recombinant protein identification and purification using, e.g., an anti-FLAG antibody.

As used herein, the term “chimeric protein” refers to two or more coding sequences obtained from different genes, that have been cloned together and that, after translation, act as a single polypeptide sequence. Chimeric proteins are also referred to as “hybrid proteins.” As used herein, the term “chimeric protein” refers to coding sequences that are obtained from different species of organisms, as well as coding sequences that are obtained from the same species of organisms.

As used herein, the term “protein of interest” refers to the protein whose expression is desired within the fusion protein. In a fusion protein, the protein of interest will be joined or fused with another protein or protein domain, the fusion partner, to allow for enhanced stability of the protein of interest and/or ease of purification of the fusion protein.

As used herein, the term “target protein” refers to proteins that are the target of post translational, covalent addition of Ub or Ubls by a complex of proteins, which in the Ub system are named E1, E2 and E3. One or more small molecule, protein, nucleic acid or other compounds may be used to target or modify the addition of Ub and/or Ubls to target proteins as taught in the assay disclosed herein. The assay may even be a high throughput assay in which a library of compounds is tested for modification to, e.g., the formation of the intermediate E2 complex between UbcH8 and ISG15 and/or the intermediate in which UbcH8 interacts with Ub.

Several steps are involved in ubiquitin (Ub) conjugation and protein degradation. First, Ub is activated by a ubiquitin-activating enzyme (E1) in an ATP dependent manner. Activation involves binding of the C-terminus of Ub to the thiol group of a cysteine residue of E1. Activated Ub is subsequently transferred to one of several Ub-conjugating enzymes (E2). Each E2 has a recognition subunit which allows it to interact with proteins carrying a particular degradation signal. E2 links the Ub molecule through its C-terminal glycine to an internal lysine of the target protein. Different ubiquitin-dependent proteolytic pathways employ structurally similar, but distinct, E2s, and in some instances, accessory factors known as ubiquitin-ligases or E3s, are required to work in conjunction with E2s for recognition of certain substrates. More than one Ub molecule may be needed to ubiquinate a target protein which is subsequently recognized and degraded by a proteasome. After degradation, Ub is released and reused. A parallel, non-coress-reacting set of enzymes has been theorized for Ub-like proteins, however, the Ub complex proteins E1, E2 and E3 (the Ub proteosome) do not cross-activate and add Ub-like proteins to protein degradation target proteins.

Prior to activation, Ub is usually expressed as a protein precursor composed of an N-terminal ubiquitin and a C-terminal extension protein (CEP) or as a polyubiquitin protein with Ub monomers attached head to tail. CEPs have characteristics of a variety of regulatory proteins; most are highly basic, contain up to 30% lysine and arginine residues, and have nucleic acid-binding domains. The fusion protein is an important intermediate which appears to mediate co-regulation of the cell's translational and protein degradation activities, as well as localization of the inactive enzyme to specific cellular sites. Once delivered, C-terminal hydrolases cleave the fusion protein to release a functional Ub.

The E2s are important for substrate specificity in several UCS pathways. All E2s have a conserved ubiquitin conjugation (UBC) domain of approximately 16 kD, at least 35% identity with each other, and contain a centrally located cysteine residue which is necessary for ubiquitin-enzyme thiolester formation. A highly conserved proline-rich element is located N-terminal to the active cysteine residue. Structural variations outside of this conserved domain are used to separate the E2 enzymes into classes. The E2s of class 1 (E2-1) consist of the conserved UBC domain and include yeast E2-1 and UBCs 4, 5, and 7. These E2s are thought to require E3 to carry out their activities. UBC7 has been shown to recognize ubiquitin as a substrate and to form polyubiquitin chains in vitro. E2s of class 2 (E2-2) have various unrelated C-terminal extensions that contribute to substrate specificity and cellular localization. The yeast E2-2 enzymes, UBC2 and UBC3, have highly acidic C-terminal extensions that promote interactions with basic substrates such as histones. Yeast UBC6 has a hydrophobic signal-anchor sequence that localizes the protein to the endoplasmic reticulum.

Abnormal activities of the UCS are implicated in a number of diseases and disorders. These include, e.g., cachexia (Llovera, M. et al. (1995) Int. J. Cancer 61: 138-141), degradation of the tumor-suppressor protein, p53 (Ciechanover, supra), and neurodegeneration such as observed in Alzheimer's disease (Gregori, L. et al. (1994) Biochem. Biophys. Res. Commun. 203: 1731-1738). Since ubiquitin conjugation is a rate-limiting step in antigen presentation, the ubiquitin degradation pathway may also have a critical role in the immune response (Grant E. P. et al. (1995) J. Immunol. 155: 3750-3758).

The discovery of new ubiquitin-conjugating-like protein and the polynucleotides encoding it satisfies a need in the art by providing new compositions which are useful in the diagnosis, treatment, and prevention of cancer, and developmental, immune and neuronal disorders.

As described hereinabove, the post-translational covalent conjugation of ubiquitin (Ub) or ubiquitin-like proteins (Ubls) is a major eukaryotic regulatory mechanism that have, heretofore, been shown to parallel one another but not overlap. Conjugation of Ub requires an enzymatic cascade involving an E1 activating enzyme, E2 conjugating enzymes, and E3 ligases. The conjugation pathways of several Ubls have been shown to parallel the Ub pathway, but to use different E1, E2 and E3 enzymes. Both the synthesis of the Ubl ISG15 and its conjugation to proteins are induced by interferon. The only characterized component of the ISG15 conjugation cascade is the E1 enzyme, UbelL, which activates ISG15, but not Ub. Disclosed herein are the isolation, purification and characterization of the E2 enzyme that functions in the ISG15 conjugation pathway. Surprisingly, UbcH8, a human E2 enzyme for ISG15 has already been shown to function in Ub conjugation. Further, the inventors demonstrate that two HECT E3 enzymes that function with UbcH8 in Ub conjugation can also function with UbcH8 in ISG15 conjugation. Thus, the pathways for Ub and ISG15 conjugation converge downstream of the E1 enzymes, at the UbcH8 E2 enzyme. This convergence raises the possibility that the alternative conjugation of a set of proteins to Ub or to ISG15 is regulated by interferon.

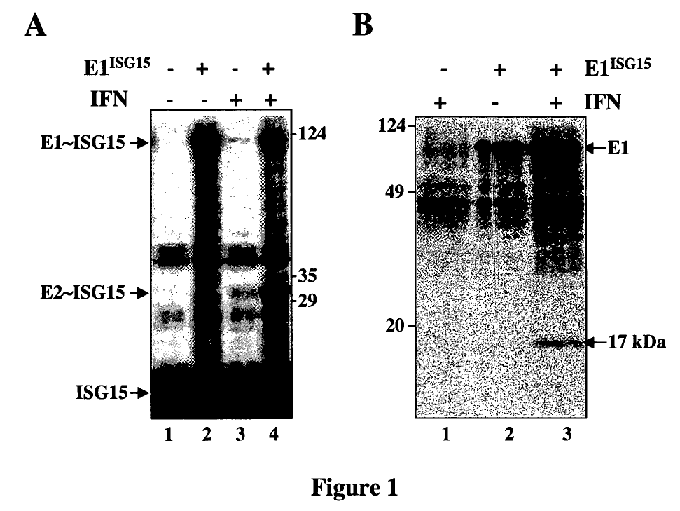

Conjugation of ISG15 to target proteins occurs in interferon (IFN)-treated human A549 human lung cells A549, indicating that these cells contain the ISG15 conjugating enzymes. To test for the presence of an E2 enzyme for ISG15, an extract from these cells was incubated with ³²P-ISG15 in the absence or presence of added UbelL (denoted as E1^{ISG15}) (FIG. 1A, lanes 3 and 4). A labeled 32 kDa product was observed. Several lines of evidence indicate that this 32 kDa species corresponds to the thioester-linked conjugate of ISG15 with its E2 enzyme. First, this species was sensitive to the reducing agent dithiothreitol (DTT) (data not shown), as expected for thioester-linked conjugates. Second, the molecular weight of the protein that is conjugated to the 15 kDa ISG15 is approximately 17 kDa, which is similar to the molecular weights of the E2s for both Ub and other Ubls. Third, formation of the labeled 32 kDa species was stimulated approximately 20-fold by the addition of E1^{ISG15}, indicating that it is likely the downstream product of a reaction involving ISG15 and its E1. The labeled 32 kDa species was not observed using extracts from A549 cells that had not been treated with IFN (lanes 1 and 2), indicating that the E2 enzyme activity was itself induced by IFN.

To purify the putative ISG15-specific E2 enzyme, GST-ISG15 and E1^{ISG15} were incubated with an extract from IFN-treated A549 cells, resulting in the formation of GST-ISG15˜E1 and GST-ISG15˜E2 conjugates. The conjugates were affinity selected using glutathione Sepharose. The thioester bonds between ISG15 and E1 and E2 were cleaved using DTT, and the eluted material was subjected to denaturing gel electrophoresis (FIG. 1B. lane 3). Coomassie blue staining detected a protein species of 17 kDa. The putative E2 species was not detected in the absence of added E1^{ISG15} (lane 1), nor when using extracts from cells not treated with IFN (lane 2). The identity of the 17 kDa species was determined by Edman degradation sequencing of the shortest peptide generated by trypsin digestion. The sequence, AEEFTLR, was identical to a sequence in UbcH8 (amino acids 149-155), a human E2 enzyme for Ub.

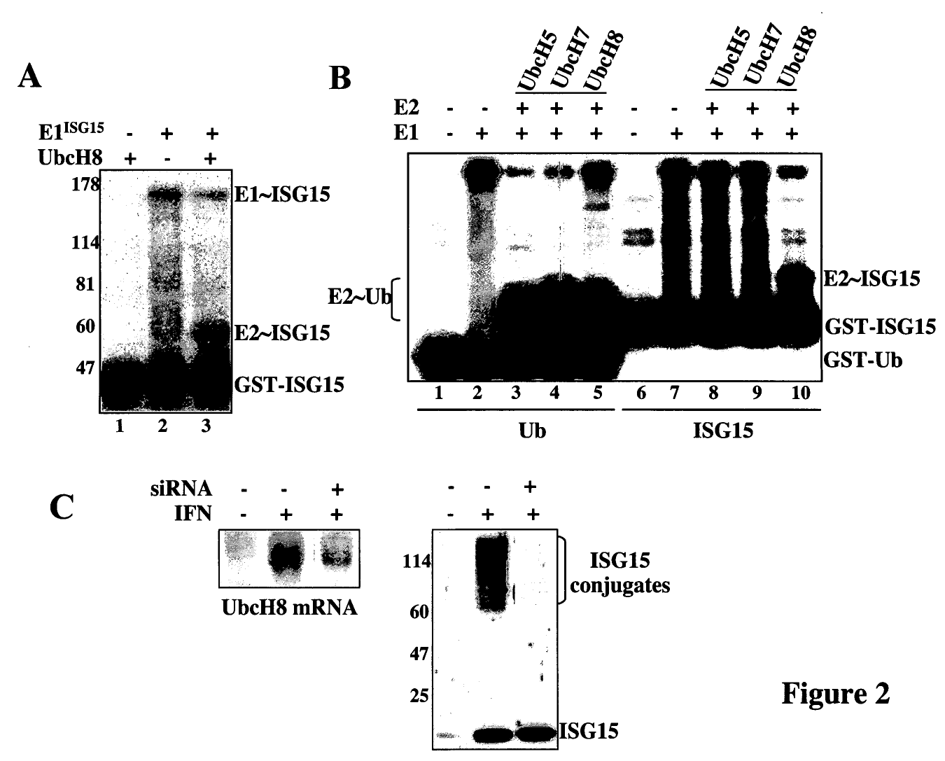

To test whether UbcH8 functions as an E2 enzyme for ISG15 in vitro, GST-³²P-ISG15 was incubated with purified UbcH8 in the absence or presence of E1^{ISG15} (FIG. 2A). ISG15 was covalently linked to UbcH8 only in the presence of E1^{ISG15} (lane 3), and the amount of ISG15 linked to E1^{ISG15} decreased concomitantly with the increase in the amount of ISG15 linked to UbcH8 (compare lanes 2 and 3). The UbcH8˜ISG15 conjugate was sensitive to DTT (data not shown). These results indicate that ISG15 was transferred from E1^{ISG15} to UbcH8, and that UbcH8 functions as an E2 for ISG15. To determine whether other E2s that are closely related to UbcH8 can also function as E2s for ISG15, we tested the activity of UbcH5b (56% similarity to UbcH8) and UbcH7 (72% similarity to UbcH8), both of which function efficiently as E2s for Ub (FIG. 2B, lanes 2-5). No E2˜ISG15 conjugates were observed with UbcH5b (compare lanes 8 and 10), and a low amount of conjugates was observed with UbcH7 (˜5% of that of UbcH8, as determined by a long exposure of the gel of FIG. 2B). The in vitro results indicate that UbcH8 is a primary E2 enzyme for ISG15.

To determine whether UbcH8 is also a primary 1SG15-specific E2 in vivo, RNA interference studies were conducted in IFN-treated HeLa cells. A double-stranded short interfering RNA (siRNA) was used directed against the UbcH8 MRNA sequence that is not identical to a sequence in any other predicted human mRNA, including the closely related UbcH7 mRNA. The UbcH8-specific siRNA reduced the amount of UbcH8 mRNA by 80-90%, and blocked IFN-induced ISG15 conjugation to a similar extent (FIG. 2C). Another siRNA directed against a different region of the UbcH8 mRNA sequence had the same effect on UbcH8 mRNA and ISG15 conjugation, and Northern and RT-PCR analyses showed that UbcH7 mRNA was not affected by either siRNA (data not shown). Northern analysis (FIG. 2C) also verified that UbcH8 mRNA was induced by IFN, as previously reported, which is consistent with our results that the E2 activity for ISG15 was only detected in A549 cells after IFN treatment (FIG. 1). Therefore, UbcH8 is an IFN-induced protein that serves as the primary E2 enzyme for ISG15 both in vitro and in vivo.

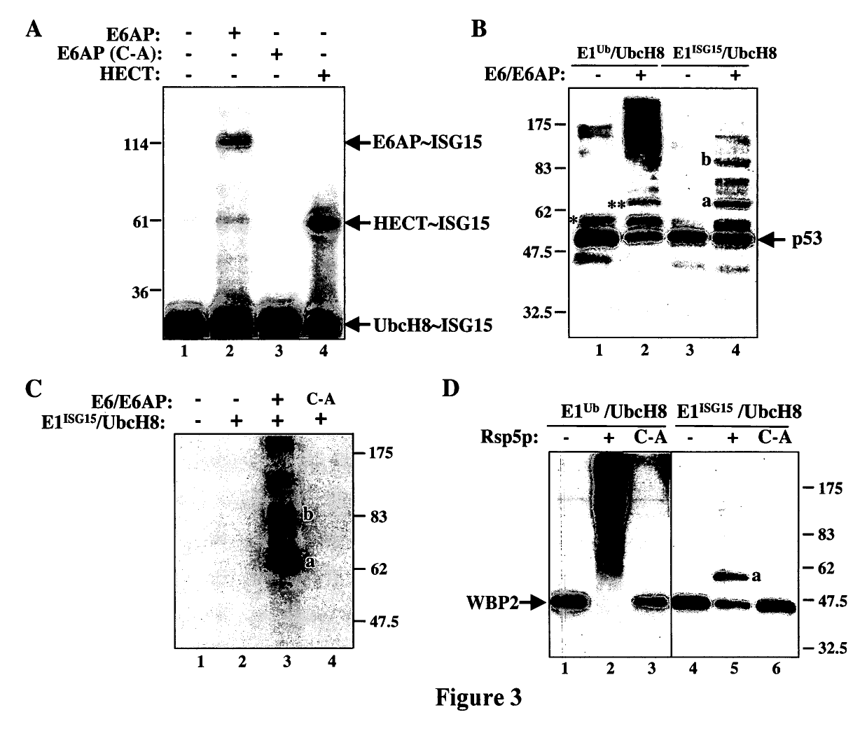

Because UbcH8 functions as an E2 for several E3 enzymes in Ub conjugation, it was next determined whether a Ub E3 would also function with UbcH8 in ISG15 conjugation. E6AP, a HECT E3, catalyzes the conjugation dependent on formation of a thioester intermediate between Ub and the active-site cysteine of the E6AP HECT domain. As shown in FIG. 3A (lane 2), E6AP formed a thioester intermediate with ISG15 in the presence of E1^{ISG15} and UbcH8, and thioester formation did not occur with a mutant E6AP in which the active-site cysteine was replaced by alanine (C-A, lane 3). The isolated 360 amino acid HECT domain also formed a thioester with 18015 (lane 4), as shown previously for Ub, and the E6AP˜ISG15 conjugates were sensitive to DTT (data not shown).

The best characterized function of E6AP is the ubiquitination of p53 in the presence of the human papillomavirus (HPV) E6 protein. Two assays established that E6AP can catalyze UbcH8-dependent conjugation of ISG15 to p53. In the first assay, immunopurified, baculovirus-expressed p53 was incubated with Ub or ISG15 in the presence of UbcH8 and the cognate E1 enzyme, and the reaction products were treated with DTT and analyzed by immunoblotting with anti-p53 antibody (FIG. 3B). The purified p53 contained a minor species that may represent mono-ubiquinated p53 (denoted by * in lane 1). High molecular weight Ub conjugates were detected in the presence of E6/E6AP (lane 2), as well as a species (denoted as **) corresponding in molecular weight to the addition of two molecules of Ub. In the ISG15 reaction, two predominant species of modified p53 were observed (FIG. 3B, lane 4), corresponding in molecular weight to the addition of one and two molecules of ISG15 (denoted as a and b, respectively). To verify that the “a and b” species contained both ISG15 as well as p53 in a covalent complex, a reaction was carried out using ³²P-ISG15, followed by immunoprecipitation with anti-p53 antibody. The predominant labeled p53 species corresponded to the molecular weights of “a and b” from the previous study (FIG. 3C, lane 3). The formation of these ISG15-p53 conjugates required both E6 and E6AP, and the active-site mutant of E6AP (C-A) did not support conjugation (FIG. 3C, lanes 2 and 4). No detectable ISG15 conjugation was observed if either E6 or E6AP was omitted from the reaction (data not shown).

To determine whether other HECT E3 enzymes can also conjugate ISG15 to a target protein, the activity of the Rsp5p E3 enzyme to conjugate ISG15 to WBP2, a protein that binds to the WW domains of Rps5p and Rsp5p homologs was determined. FLAG-tagged WBP2 was purified and the reaction products were analyzed by immunoblotting with anti-FLAG antibody (FIG. 3D). Rsp5p catalyzed the efficient polyubiquitination of WBP2 in the presence of E1^{Ub} and UbcH8, while the active-site mutant (C-A) was inactive (lanes 1-3). Wild-type Rsp5p also catalyzed the conjugation of ISG15 to WBP2 in the presence of E1^{ISG15} and UbcH8 (lanes 4-6). The molecular weight of the predominant WBP2-ISG15 conjugate (denoted as a) corresponded to the addition of a single molecule of ISG15, although faint higher molecular weight bands were detected that might correspond to conjugates containing two-to-four ISG15 molecules.

Studies using ³²P-ISG15 verified that the predominant conjugate shown in lane 5 contained ISG15 (not shown). Together, the results with E6AP and Rsp5p strongly suggest that multiple HECT E3s support ISG15 conjugation. Further, only one or two ISG15 protein molecules were conjugated to the test substrates in vitro, under conditions where Ub was being conjugated in polyubiquitin chains. The conjugates containing two ISG15 molecules may represent either two ISG15s linked in a chain to a single lysine of the target protein, or single ISG15 molecules linked to different lysines of the target protein. It will clearly be important to establish the nature of ISG15 conjugates formed both in vitro and in vivo.

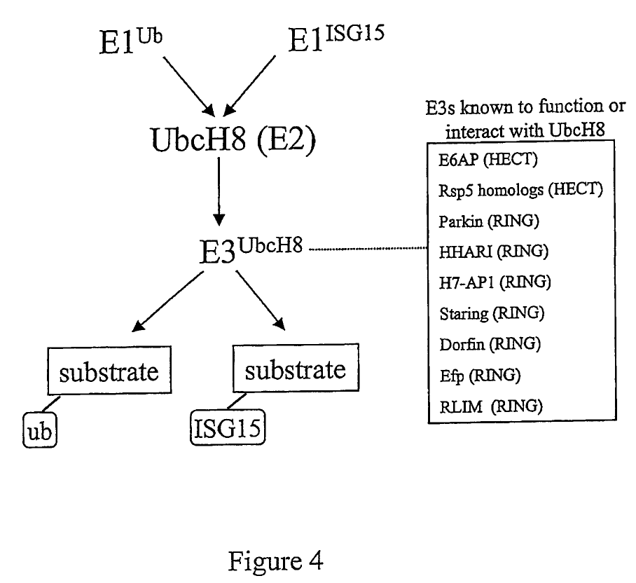

Our results demonstrate that the pathways for Ub and ISG15 conjugation converge downstream of the E1 enzymes, at the UbcH8 E2 enzyme (FIG. 4). While conjugation pathways for at least ten Ubls have been identified in various organisms, this is the first documentation of the convergence of a Ubl conjugation pathway with a Ub conjugation pathway. The E1^{ISG}15 enzyme appears to function selectively with the UbcH8 E2 enzyme, both in vitro and in vivo, whereas the E1 enzyme for Ub functions with multiple E2 enzymes. It will be of interest to determine how ISG15-charged E1^{ISG15} distinguishes UbcH8 from other E2s, particularly the most closely related E2 enzyme, UbcH7. The data included herein demonstrates that the ISG15-charged UbcH8 can function I with two HECT E3 enzymes that function with UbcH8 in Ub conjugation. While no E3s have yet been confirmed to function with ISG15 in vivo, it is likely that other E3 enzymes that function with UbcH8 (E3^{Ubc8} enzymes), RING as well as HECT E3s, will also function in ISG15 conjugation in vitro (FIG. 4).

Several of the E3 enzymes have been implicated in important diseases, including E6AP (Angelman's syndrome and cervical cancer), Parkin (Parkinson's disease) and Efp (breast cancer). Because E3 enzymes that function with UbcH8 often also function with the closely related UbcH7 enzyme, the E3^{UbcH7} enzymes are also good candidates for functioning with ISG15.

The convergence of the Ub and ISG15 conjugation pathways suggests that proteins that are targets for ISG15 conjugation (e.g., STAT1, ERK1, serpin 2a) are also targets for Ub conjugation. Whereas Ub conjugation often targets proteins for degradation, this does not appear to be the case for ISG15 conjugation. Because both ISG15 and UbcH8 expression are induced by IFN, ISG15 modification may prevent Ub-mediated proteolysis of a set of proteins during the IFN response. In the absence of IFN, the E3 enzymes that function with UbcH8 (FIG. 4) may conjugate Ub to protein targets by using E2s that are related to UbcH8, for example, UbcH7, which is constitutively expressed in the absence of IFN. After IFN treatment, the same E3 enzymes would be presented with high levels of ISG15-charged UbcH8, and as a result may predominately conjugate ISG15 to the same protein targets. In addition to possibly stabilizing target proteins, ISG15 conjugation may also modulate their function. Such a switch from Ub to ISG15 conjugation occurring after IFN treatment would be expected to have multiple, profound effects on cellular functions.

Materials and Methods

Preparation of A549 cell extracts. Crude cell extracts from A549 cells, either untreated or treated with 1000 units/ml of IFN-β (Berlex Co.), were prepared as described previously. The extracts were centrifuged at 12,000×g for 10 minutes, and the supernatant was passed through a glutathione Sepharose column to remove cellular proteins that bind directly to this matrix. The flow through was applied to an SP Sepharose column to remove activities that interfered with optimum ISG15 conjugation. The flow through from this column was concentrated to 20 mg/ml using Centricon-10 filters (Amicon), and then used for the purification of the E2 enzyme for ISG15, as described hereinbelow.

Protein expression and purification. E1^{Ub} and E1^{ISG15} were expressed as GST-fusions using baculovirus vectors (Pharmingen and Invitrogen) in High Five insect cells (Invitrogen, and ubiquitin and ISG15 were expressed as GST-fusions using the pGEX2TK vector (Amersham) in E. coli strain BL21. UbcH5b, UbcH7, UbcH8, E6AP HECT domain (residues 495 to the C-terminus) and E6AP HECT domain containing the C820A mutation were expressed in E. coli strain BL21 as GST-fusions using the pGEX4T1 vector (Amersham Biosciences). These proteins were purified by glutathione affinity chromatography, and where indicated, the GST-fusions were further purified by size exclusion chromatography following thrombin cleavage to remove GST. FLAG-tagged WBP2 protein was expressed in E. coli, as a GST-fusion using the pGEX-6p vector (Amersham), and full-length E6AP, the E6AP C-A mutant, and HPV33 E6 were expressed in High Five insect cells as GST fusions using a baculovirus vector. After glutathione affinity chromatography, the GST moiety of the latter three fusions was removed by digestion with PreScission protease (Amersham). Human p53 was expressed using a baculovirus vector in High Five cells and immunopurified using monoclonal antibody pAb421. Proteins were stored at −80 at concentrations of 0.5-15 mg/ml.

Thioester and Ub and ISG15 conjugation assays. Ub and ISG15 thioester assays (FIGS. 2 and 3) used 200 μg purified E1^{Ub} or E1^{ISG15} purified E2 enzyme, and where indicated, 2-5 μg E6AP protein, Reactions that utilized ³²P-labeled Ub or ISG15 contained approximately 400 ng Ub or ISG15 (10⁶ cpm); these proteins were labeled as described by Yuan and Krug. Reactions contained 25 mM Tris (pH 7.5), 50 mM NaCl, 10 mM MgCl₂, 5 mM ATP, and 0.1 mM DTT, and were incubated at 25° for 10 minutes. They were stopped with SDS-PAGE loading buffer lacking DTT and analyzed by SDS-PAGE and autoradiography.

Substrate conjugation assays (FIG. 3) using unlabeled Ub or ISG15 contained 200 ng Ub or ISG15, 100 ng E1^{Ub} or E1^{ISG15}), 1.5 μg UbcH8, 100 ng E6AP or Rps5p protein, and 2 μg purified p53 or FLAG-WBP2, and where indicated, 40 ng HPV33 E6 protein. Reactions contained 25 mM Tris (pH 7.5), 50 mM NaCl, 10 mM MgCl₂, 5 mM ATP and 0.1 mM DTT, and were incubated at 25° for 30 minutes. Reactions were stopped with DTT-containing SDS-PAGE loading buffer and analyzed by immunoblotting, using either anti-p53 antibody (Ab6, Calbiochem) or anti-FLAG antibody (Sigma). Reactions containing ³²P-labeled ISG15 (FIG. 3C) were done under the same conditions, substituting 106 cpm (400 ng) ³²P-labeled ISG15. Reactions were stopped by dilution into buffer containing 10 mM EDTA, p53 was immunoprecipitated with anti-p53 antibody (Ab6, Calbiochem), and the immunoprecipitates were analyzed by SDS-PAGE and autoradiography.

FIGS. 1A and 1B are gels that show the identification of the E2 enzyme for ISG15 conjugation. In FIG. 1A extracts from IFN-treated (lanes 3 and 4) or untreated (lanes 1 and 2) A549 cells were incubated with ³²P-1SG15 protein in the absence (lanes 1 and 3) or presence (lanes 2 and 4) of E1^{ISG15} in a final volume of 50 μl for 30 minutes at 27° C. The protein products were resolved by electrophoresis on a 10% polyacrylamide gel. The positions of E1˜ISG15 and E2˜1SG15 adducts are denoted, with positions of molecular weight markers shown on the right. In FIG. 1B the affinity purification of the E2 enzyme for ISG15 is shows. Cell extract (15 mg protein) from IFN-treated (lanes 1 and 3) or untreated (lane 2) A549 cells were incubated with (GST-ISG15 (2 mg) in either the absence (lane 1) or presence (lane 2 and 3) of E1^{ISG15} (500 μg˜ in a final volume of 1 ml for 30 minutes at 27° C. under the thioester reaction conditions described hereinabove. The reaction products were affinity selected on glutathione Sepharose, and thioester bonds were cleaved by DTT treatment. The eluted proteins were subjected to electrophoresis on 15% polyacrylamide gels, followed by Coomassie blue staining. The 17 kDa protein in lane 3 was digested with trypsin, and the smallest tryptic peptide was sequenced by automated Edman degradation.

FIGS. 2A, 2B and 2C demonstrate that UhCH8 functions as the predominant E2 enzyme for ISG15 both in vitro and in vivo. In FIG. 2A ³²P-GST-ISG15 was incubated in the presence (lanes 1 and 3) or absence (lane 2) of UbcH8, and in the presence (lane 2 and 3) or absence of (lane 1) of E1^{ISG15}. The thioester-linked E1^{ISG15} and UbcH8 adducts are indicated. In FIG. 2B, either ³²P-GST-Ub (lanes 1-5) or P-GST-ISG15 (lanes 6-10) was incubated in the absence (lanes 1 and 6) or presence of E1^{Ub} (lanes 2-5) or E1^{ISG15} (lanes 7-10), and in the absence of E2 (lanes 2 and 7) or in the presence of the indicated E2 enzymes (lanes 3-5, 8-10). In FIG. 2C, shows the results of RNA interference studies in vivo in which HeLa cells were either mock transfected (−siRNA) or transfected with an siRNA (20 nM) directed against bases 28-49 of the open reading frame of human UbcH8 mRNA (+siRNA lanes). Twenty four hours later, the cells were left untreated (−IFN lanes) or were treated with IFN-β (1000 units/ml) (+IFN lanes). After an additional 24 hour incubation, the cells were collected. RNA was analyzed for UbcH8 mRNA by Northern analysis (left panel), and proteins were analyzed by immunoblotting with ISG15 antiserum (right panel). Each lane of the Northern blot contained 12 μg total RNA, and the presence of equal amounts of RNA in each lane was confirmed by ethidium bromide staining of 28S ribosomal RNA. The same results were obtained using a second siRNA that was directed against bases 239-258 of the open reading frame of UbcH8 mRNA, and an siRNA directed against a sequence in the mRNA for green fluorescent protein did not decrease either UbcH8 mRNA or ISG15 conjugation (not shown).

FIGS. 3A, 3B, 3C and 3D demonstrate that two HECT E3s function with ISG15 in vitro. In FIG. 3A, ISG15-thioester formation with E6AP is shown. All reaction mixtures contained ³²P-ISG15, E1^{ISG15} and UbcH8. Where indicated, full-length E6AP (lane 2), its active site (C-A) mutant (lane 3), or the 360 amino acid E6AP HECT domain (lane 4) was added. In FIG. 3B, HPV E6/E6AP-dependent conjugation of ISG15 to p53 is shown. Baculovirus-expressed immunopurified p53 was incubated with either Ub, E1^{Ub}, and UbcH8 (lanes 1 and 2), or ISG15, E1^{ISG15} and UbcH8 (lanes 3 and 4). Reactions either lacked (lanes 1 and 3) or contained (lanes 2 and 4) purified HPV P6 and E6AP. Reactions were stopped with a buffer containing SDS and DTT, and the products were analyzed by immunoblotting with anti-p53 antibody. Mono- and di-ubiquinated p53 species are denoted as * and **, respectively. Products corresponding in size to mono-ISG15 and di-ISG15 conjugates to p53 are denoted by a and b, respectively. In FIG. 3C, ³²P-ISG15 was incubated with purified p53 either in the absence (lane 1) or presence (lanes 2-4) of E1^{ISG15} and UbcH8. Reactions either lacked (lanes 1 and 2) or contained HPV33 E6 and E6AP (lane 3), or contained HPV E6 and the C-A mutant of E6AP (lane 4). Reactions were stopped, p53 was immunoprecipitated, and the immunoprecipitates were analyzed by SDS-PAGE and autoradiography. Products corresponding in size to mono-ISG15 and di-ISG15 conjugates to p53 are denoted by a and b, respectively. FIG. 3D shows the results from using purified FLAG-WBP2 protein was incubated with E1, E1^{Ub} and UbcH8 (lanes 1-3) or ISG15, E1^{ISG15} and UbcH8 (lanes 4-6), in the absence (lanes 1 and 4) or presence of Rsp5p (lanes 2 and 5) or in the presence the C-A mutant of Rsp5p (lanes 3 and 6). Reactions were stopped with a buffer containing SDS and DTT, and the products were analyzed by immununoblotting with anti-FLAG antibody. The product corresponding to a mono-ISG15 conjugate to WBP2 is denoted (a).

FIG. 4 shows the intersection of the conjugation pathways for Ub and ISG15 conjugation converge downstream of the E1 enzymes, at the UbcH8 E2 enzyme. As shown herein, the two E3 enzymes that function with UbcH8 can alternatively conjugate Ub or ISG15 to substrates in vitro. The box on the right lists the E3 enzymes that have been shown to function with UbcH8 (E3 UbcH8 enzymes).

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Schwartz, D.C. & Ilochstrasser, M. A superfamily of protein tags: ubiquitin, SUMO and related modifiers. Trends Biochem S W 28, 32 1-8 (2003).
2. Kim, K. I. & Zhang, D. E. ISG15, not just another ubiquitin-like protein. Biochem BiophysRes Commun 307, 43 1-4 (2003).
3. Yuan, W. & Krug, R. M. Influenza B virus NS1 protein inhibits conjugation of the interferon (IFN)-induced ubiquitin-like ISG15 protein. EMBO J 20, 362-71 (2001).
4. Kumar, S., Kao, W. H. & Howley, P. M. Physical interaction between specific E2 and Hect E3 enzymes determines functional cooperativity. J Biol Chem. 272, 13548-54 (1997).
5. Loeb, K. K. & Haas, A. L. Conjugates of ubiquitin cross-reactive protein distribute in a cytoskeletal pattern. Mol Cell Biol 14, 840 8-19 (1994).
6. Pickart, C. M. Mechanisms underlying ubiquitination. Annu Rev Biochem 70, 503-33 (2001).
7. Jensen, J. P., Bates, P. W., Yang, M., Vierstra, K. D. & Weissman, A. M, Identification of a family of closely related human ubiquitin conjugating enzymes. J Biol. Chem. 270, 30408-14 (1995).
8. Nuber, U., Schwarz, S., Kaiser, P., Schneider, R. & Scheffluer, M. Cloning of human ubiquitin-conjugating enzymes IThCH6 and UbcTJ7 (22-Fl) and characterization of their interaction with 26-AlP and RSP5. J Biol. Chem. 271, 2795-800 (1996).
9. Elbashir, S. M. et al. Duplexes of 21-nucleotide RNAs mediate RNA interference in cultured mammalian cells. Nature 411, 494-8 (2001).
10. Nyman, T. A., Matikainen, S., Sareneva, T., Julkunen, I. & Kalkkinen, N. Proteome analysis reveals ubiquitin-conjugating enzymes to be a new family of interferon-alpha-regulated genes. Eur J Biochem 267, 4011-9 (2000).
11. Chin, L. S., Vavalle, J. P. & Li, L. Staring, a novel 23 ubiquitin-protein ligase that targets syntaxin I for degradation. J Biol Chem. 277, 3507 1-9 (2002).
12. Zhang, Y. et al. Parkin functions as an 22-dependent ubiquitin-protein ligase and promotes the degradation of the synaptic vesicle-associated protein, CDCrel-1. Proc Natl Acad Sci USA 97, 133 54-9 (2000).
13. Niwa, J. et al. A novel centrosonual ring-finger protein, dorfin, mediates ubiquitin ligase activity. Biochem Biophys Res Commun 281, 706-13 (2001).
14. Urano, T. et al. Bfp targets 14-3-3 sigma for proteolysis and promotes breast tumour growth. Nature 417, 871-5 (2002).
15. Kramer; 0. II. et al. The histone deacetylase inhibitor vaiproic acid selectively induces proteasomal degradation of HDAC2. EMBO J22, 3411-20 (2003).
16. Moynihan, T. P. et al. The ubiquitin-conjugating enzymes UbcH7 and IJbCH8 interact with RING finger/IBR motif containing domains of HHAR1 and H7-AP 1. J Biol Chem. 274, 30963-8 (1999).
17. Scheffner, M., Nuber, U. & Huibregtse, I. M. Protein ubiquitination involving an 21-22-E3 enzyme ubiquitin thioester cascade. Nature 373, 8 1-83 (1995).
18. Huang, L. at al. Structure of an E6AP-UbcH7 complex: insights into ubiquitination by the B2-B3 enzyme cascade. Science 286, 132 1-6 (1999).
19. Huibregtse, J. M., Maki, C. G. & Howley, P. M. in Ubiquitin and the biology of the cell (eds. Peters, S. M. & Finley, D.) 324-343 (Plenum Publishing Corp., New York, N.Y., 1998).
20. Pirozzi, 0. et al. Identification of novel human WW domain-containing proteins by cloning of ligand targets. J. Biol. Chem. 272, 14611-6 (1997).
21. Kishino, T., Lalande, M. & Wagstaff, I. UBESA/B6-AP mutations cause Angelinan syndrome. Nature Genet 15, 70-73 (1997).
22. Matsuura, T. et al. De nova truncating mutations in E6-AP ubiquitin-pratein ligase gene ˜IJBE3A) in Angelinan syndrome. Nat Genet 15, 74-7 (1997).
23. Scheffner, M., Huibregtse, S. M., Vierstra, R. D. & Hawley, P. M. The 1ff V-16 1E6 and E6-AP Complex Functions as a Ubiquitin-Protein Ligase in the Ubiquitination of p53. Cell 75, 495-505 (1993).
24. Shimura, H. et al. Familial Parkinson disease gene product, parkin, is a ubiquitin-protein ligase. Nat Genet 25, 302-5 (2000).
25. Tanaka, K. et al. Parkin is linked to die ubiquitin pathway. J Mol Med 79, 482-94 (2001).
26. Malakhov, M. P. et al. High-throughput immunablatting. Ubiquitiin97like protein 15015 modifies key regulators of signal transduction. J Biol Chem 278, 16608-13 (2003).
27. Hamerman, S. A., et al. Serpin 2a is induced in activated macrophages and conjugates to a ubiquitin homolog. J Immunol 168, 2415-23 (2002).
28. Farrell, P. J., Broeze, R. S. & Lengyel, P. Accumulation of an mRNA and protein in interferon-treated Ebrlich ascites tumour cells. Nature 279, 523-5 (1979).
29. Korant, B. D., Blonistrom, D. C., Jonak, J. K. & Knight, E., Jr. Interferon-induced proteins. Purification and characterization of a 15,000-dalton protein from human and bovine cells induced by interferon. J Biol C92hem 259, 14835-9 (1984).
30. Friedman, P. N., Kern, S. B., Vogeistein, B. & Prives, C. Wild-type, but not mutant, human p53 proteins inhibit the replication activities of simian virus 40 large tumor antigen. Proc Natl Acad Sci USA 87, 9275-9 (1990).

1. A method of selecting a compound that modulates the conjugation of Ubiquitin and Ubiquitin-like proteins to a target protein comprising the step of:
contacting the compound with a Ubiquitin-like protein conjugating enzyme that conjugates a Ubiquitin or a Ubiquitin-like protein to the target protein; and
detecting the effect of the compound on the Ubiquitin-like protein conjugating enzyme.

2. The method of claim 1, wherein the Ubiquitin-like protein conjugating enzyme comprises UbcH8.

3. The method of claim 1, wherein the Ubiquitin-like protein conjugating enzyme comprises an interferon-inducible ubiquitin conjugation E2 complex.

4. The method of claim 1, wherein expression of the Ubiquitin-like protein conjugating enzyme is upregulated by interferon.

5. The method of claim 1, further comprising adding a complete protein degradation proteosome and detecting the level of the target protein in the presence or absence of the compound.

6. The method of claim 1, wherein the effect detected is protein degradation, protein binding, protein labeling, protein presence, ubiquitin conjugation, ubiquitin-like protein conjugation, thioester-linkage between the Ubiquitin or Ubiquitin-like protein and the Ubiquitin-like conjugating enzyme and combinations thereof.

7. The method of claim 1, wherein the step of contacting is performed in vitro.

8. The method of claim 1, wherein the Ubiquitin-like protein conjugating enzyme comprises an isolated and purified fusion UbcH8 protein.

9. The method of claim 1, wherein the Ubiquitin-like protein conjugating enzyme comprises GST-UbcH8.

10. The method of claim 1, wherein the Ubiquitin or a Ubiquitin-like protein comprises an isolated and purified Ubiquitin-like fusion protein.

11. The method of claim 1, wherein the Ubiquitin or a Ubiquitin-like protein comprises GST-ISG15.

12. A method of selecting a compound that modulates the conjugation of Ubiquitin-like proteins to target proteins comprising the steps of:
contacting the compound with an Ubiquitin-like E2 enzyme and the Ubiquitin-like E1 enzyme comprising the ISG15 protein; and
detecting the effect of the compound on the interaction between the Ubiquitin-like E2 enzyme and the Ubiquitin-like E1 enzyme comprising the ISG15 protein.

13. The method of claim 12, wherein the Ubiquitin-like E2 enzyme comprises UbcH8.

14. The method of claim 12, wherein the Ubiquitin-like E2 enzyme is an interferon-inducible ubiquitin conjugation E2 complex.

15. The method of claim 12, wherein the interferon is a type I interferon.

16. The method of claim 12, wherein the Ubiquitin-like E2 enzyme conjugates Ubiquitin to a target protein.

17. The method of claim 12, wherein the Ubiquitin-like E2 enzyme conjugates a Ubiquitin-like protein to a target protein.

18. The method of claim 12, further comprising the step of adding a target protein comprising the ubiquitin-like protein that is degraded via the Ubiquitin-like or Ubiquitin protein pathway and detecting the level of the target protein in the presence or absence of the compound.

19. The method of claim 12, wherein the effect detected is protein degradation, protein binding, protein labeling, protein presence, ubiquitin conjugation, ubiquitin-like protein conjugation, thioester-linkage between the Ubiquitin or Ubiquitin-like protein and the Ubiquitin-like E2 enzyme and combinations thereof.

20. The method of claim 12, wherein the step of contacting is performed in vitro.

21. The method of claim 12, wherein the Ubiquitin-like E2 enzyme comprises an isolated and purified protein.

22. The method of claim 12, wherein the Ubiquitin-like E2 enzyme comprises GST-UbcH8.

23. The method of claim 12, wherein the ISG15 protein comprises an isolated and purified protein.

24. The method of claim 12, wherein the ISG15 comprises GST-ISG15.

25. A method of selecting a compound that modulates both the degradation of Ubiquitin and Ubiquitin-like proteins comprising the step of:
contacting the compound with the E2 enzyme HbcH8 and an ISG15 protein; and
detecting the effect of the compound on the interaction between the HbcH8 and the ISG15 proteins.

26. The method of claim 25, wherein the Ubiquitin-like protein E2 enzyme comprises GST-UbcH8.

27. The method of claim 25, wherein the ISG15 comprises GST-ISG15.

28. The method of claim 25, further comprising adding a target protein comprising the ubiquitin-like protein that is degraded via the Ubiquitin-like or Ubiquitin protein pathway and detecting the level of the target protein in the presence or absence of the compound.

29. The method of claim 25, wherein the effect detected is protein degradation, protein binding, protein labeling, protein presence, ubiquitin conjugation, ubiquitin-like protein conjugation, thioester-linkage between the Ubiquitin or Ubiquitin-like protein and the Ubiquitin-like E2 enzyme and combinations thereof.

30. A method of selecting a compound that modulates the degradation of a target protein comprising the step of:
contacting the compound with a UbcH8 and protein a Ubiquitin or a Ubiquitin-like protein; and
measuring the level of degradation of the target protein.

31. A high throughput assay for selecting a compound that modulates the conjugation of Ubiquitin and Ubiquitin-like proteins to a target protein comprising the step of:
contacting one or more compounds with a Ubiquitin-like protein conjugating enzyme that conjugates a Ubiquitin or a Ubiquitin-like protein to a target protein;
detecting the effect of the compound on the Ubiquitin-like protein conjugating enzyme; and
isolating the one or more compounds that affect the activity of the Ubiquitin-like protein conjugating enzyme.

32. An isolated protein that binds to both Ubiquitin and Ubiquitin-like proteins leading to the degradation of a protein.

33. An inhibitor of ubiquitin-like protein degradation identified by the method of claim 1.

34. An inhibitor of ubiquitin-like protein and ubiquitin degradation comprising one or more molecules that interfere with the binding of ISG15 and UbcH8.

35. An inhibitor of ubiquitin-like protein and ubiquitin degradation comprising one or more molecules that interfere with the binding of Ubiquitin and UbcH8.
